(19) United States
(12) Patent Application Publication
(10) Pub. No.: US 2013/0240756 A1
(43) Pub. Date: Sep. 19, 2013

(54) Escalator and/or Moving Walkway Rail Sterilization System

(71) Applicant: Stanley Segal, Boca Raton, FL (US)

(72) Inventor: Stanley Segal, Boca Raton, FL (US)

(21) Appl. No.: 13/419,680

(22) Filed: Mar. 14, 2012

(51) Int. Cl. A61L 2/10

(52) U.S. Cl. 250/492.1

(57) ABSTRACT

A system for sterilization of handrails having a UV light sterilization unit; a placement position for said unit, said position being proximately located to a moving escalator or walkway handrail; and activating said UV light unit, wherein said activation sterilizes said handrail continually while said handrail is in motion.

### FIELD OF INVENTION

The present invention relates to the general art of escalators and moving walkways, and to the particular field of escalator and moving walkway handrails.

### BACKGROUND OF THE INVENTION

Escalators and/or moving walkways are installed inside and or outside of buildings or structures that have multiple floors. Escalators are used in department stores, office buildings, airport terminals, subway and train stations, arenas, convention centers and hotels to move people in large numbers up or down to one floor to another floor.

Moving walkways, which are a horizontal traveling type of escalator, are used on single floor level to speed the travel of pedestrians along walkways along a horizontal or inclined plain.

The handrails associated with these assisted traveling systems serve as handgrips and are intended to prevent pedestrians from falling down or slipping.

The handrails are commonly made of rubber or a flexible composite material. Pedestrian's hands come in contact with the handrails and they deposit various bacteria, viruses and the like to the surface of the handrails that may be harmful to other pedestrians that touch the same contaminated area of the handrail. Bacteria and viruses continue to propagate on the handrails. Therefore handrails should be continually disinfected to prevent the spread of infectious microbes.

Presently, sanitary maintenance of the moving escalator and moving walkway handrails is attempting to being fulfilled by periodically cleaning the surfaces with neutral cleaning agents or antiseptic solutions. It is clear that the railing surfaces are not disinfected between the periodic cleaning intervals.

As these moving hand rails must be frequently cleaned manually, there is a problem in that the maintenance and cleaning supplies are becoming very expensive. In addition, since the cleaning work cannot positively sterilize and disinfect the handrails consistently after each persons usage travelers are exposed to hazards such as disease causing bacteria and virus infections due to the bacteria and virus existing on the handrails.

Accordingly, escalator and moving walkway handrails in both public and private structures are either left dirty, or at best, taken care of by occasional cleaning. Without proper protection and safeguards, the general public is constantly subjected to these unsanitary conditions.

Such a situation is not only costly but very detrimental from the standpoint of public safety.

### SUMMARY OF THE INVENTION

The present invention is a device for enclosing the top and two sides of escalator and or moving walkway handrails for the purpose of disinfecting and killing deposited microbes during normal operation. The device uses a UV Germicidal Lamp to kill the virus and bacteria without any foreign objects or chemicals touching the handrail. As the handrail passes through the device it passes through a chamber that contains the UV Germicidal lamp.

Exposure to the UV light causes damage to the nucleic acid of microorganisms by forming covalent bonds between certain adjacent bases in the DNA. The formation of such bonds prevents the DNA from being unzipped for replication, and the organism is unable to reproduce. In fact, when the organism tries to replicate, it dies. The close proximity of the UV Germicidal lamp to the handrails of the escalator and or moving walkways kills most microorganisms in micro seconds. The UV Germicidal lamp device is contained in a metal housing that is attached to the substructure of the escalator and or moving walkway using a mounting bracket on each side of the escalator.

### OBJECTS OF THE INVENTION

The object of the present invention is to provide a device for continuously disinfecting escalator and/or moving walkway handrails during normal operation without shutting down the unit.

Another object of the invention is to continuously disinfect and sanitize the escalator and or moving walkway handrails without any cleaning material products or substances touching or be in direct contact with the handrails.

Another object of the invention is to provide a device that eliminates the cost of cleaning supplies and materials used in the maintenance of escalator and or moving walkway handrails.

Another object of the invention is to reduce the labor costs involved in the cleaning and maintenance of escalator and or moving walkway handrails.

Another object of the invention is to prevent the spread of infectious bacteria and viruses from one person to another person when harmful microorganisms are deposited on the surface of escalator and or moving walkway handrails by persons holding on to the handrails when traveling on the escalator and or moving walkways.

In one embodiment, the present invention is a system and method for sterilization of handrails comprising:

providing an UV light sterilization unit; identifying a placement position for said unit, said position being proximately located to a moving escalator or walkway handrail;
placing said unit in said placement position; activating said UV light unit, wherein said activation sterilizes said handrail continually while said handrail is in motion.

The system provides UV light at a wavelength from 240-280 nm, preferably at a wavelength of 254 nm.

The unit provides continuous UV light.

In one embodiment, the unit provides UV light in regular pulses.

Preferably, the unit provides a degree of microbial inactivation by light intensity and exposure time at 2,000 to 8,000 μW·s/cm².

The intensity and exposure time is correlated such that said unit provides said intensity and exposure time relative to said moving handrail.

The unit is positioned such that it provides an unobstructed light pathway to said handrail, and preferably is in a position where it is not accessible to users of said handrail.

In one embodiment, the unit has one or more detectors to optimize efficiency. The detectors can include detection means to measure the level of microorganisms on said handrail and automatic adjustments of UV light duration and intensity based on said measurements.

The unit can also include means to measure the speed of said handrail and adjusts light and intensity based on handrail speed.

### BRIEF DESCRIPTION OF THE DRAWINGS

FIG. 1 is a perspective drawing of the device housing

FIG. 2 is a end view cut away drawing of the device

FIG. 3 is a perspective drawing of the device mounted on an escalator

FIG. 4 is a perspective drawing of the device mounted on a moving walkway.

### DETAILED DESCRIPTION OF THE PREFERRED EMBODIMENTS

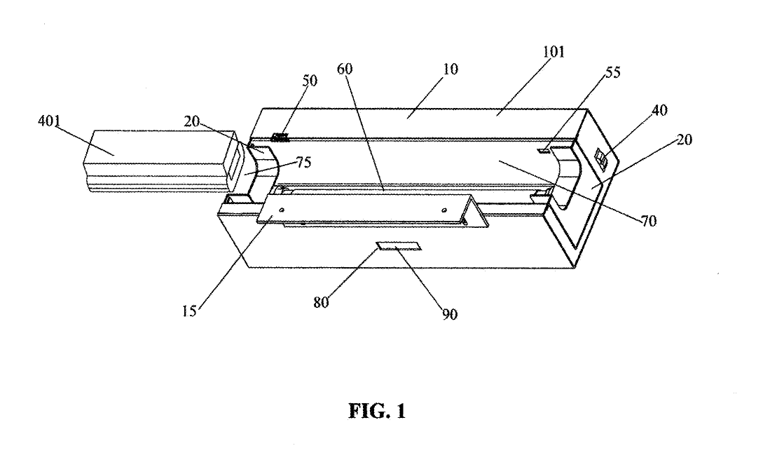

FIG. 1 shows the preferred embodiment of the UV Germicidal Lamp Housing 101 as being rectangular with a slot cavity configured to the shape of the handrail 401 and running down one side allowing the handrail to pass through and over said cavity, in close proximity to the UV Germicidal Lamp 60. The main components of the invention are the outer case 10 to house the components. Outer case 10 is positioned on or proximate to the escalator substructure 210 and or moving walkway substructure 105 with a support bracket 15. On each end of the housing 101 are the shades 20 constructed and arranged to prevent UV rays from escaping through the ends of the housing 101 as the handrail 401 passes through the device. This construction refers to the shape, configuration, and material composition to restrict the path of UV light. The UV radiation is provided by a UV Germicidal Lamp 60 surrounded by a linear parabolic reflector 70 which is made of metal with a highly polished surface to maximize and direct the UV rays onto handrail 401. The Lamp 60 is controlled by an On/Off service switch 40 to control the power during service.

The UV Germicidal Lamp Housing 101 also contains a power safety switch 50 to prevent the UV Germicidal Lamp 60 from turning on during servicing or when the housing 101 is removed from the escalator substructure.

The UV Germicidal Lamp Housing 101 also has an observation window 80 to visually check the operation of the lamp. This opening is covered by a UV lens 90 to prevent injury to servicing personnel eyes.

The UV Germicidal Lamp Housing 101 also has a motion sensor 55 that detects movement of the handrail 401. While the escalator and moving walkway handrails 401 are in motion the UV Germicidal Lamp 60 remains on. Should the escalator and moving walkway stop for any reason the UV Germicidal Lamp 60 is turned off.

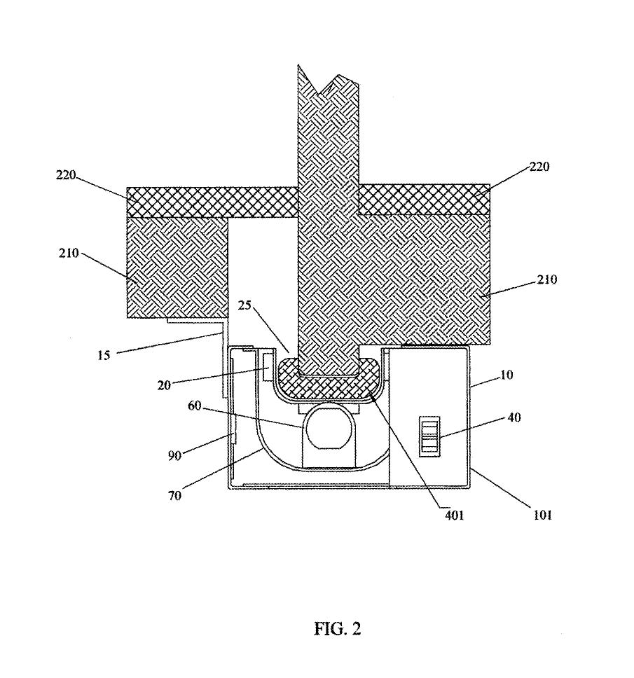

FIG. 2 shows the UV Germicidal Lamp Housing 101 cut away from one end, exposing the main components of the unit. The escalator and or moving walkway handrail 401 passes through and over the shade 20 in the configured slot area 75 in the UV Germicidal Lamp Housing 101, exposing the handrail 401 directly to the UV Germicidal Lamp 60 destroying all surface microbes. The shade 20 prevents the UV light from escaping from the UV Germicidal Lamp Housing 101 to prevent harm to service personnel. The UV linear parabolic reflector 70 is highly polished to reflect the rays towards the handrail 401. The UV Germicidal Lamp Housing 101 is mounted to the escalator and or moving walkway substructure 210 using mounting bracket 15 beneath the fascia panel 220. Power for the unit is controlled by a power switch 40.

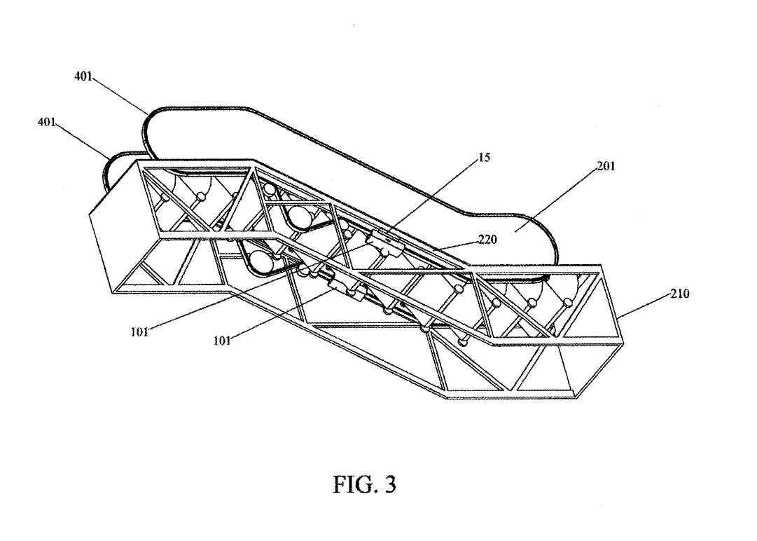

FIG. 3 shows the UV Germicidal Lamp Housing 101 mounted to the escalator substructure 210 for each handrail 401 on each side of the escalator 201 with a mounting bracket 15. The unit is positioned beneath the fascia panel 220 in the service area of the escalator 201. Every time the handrail 401 passes through the UV Germicidal Lamp Housing 101 and is exposed to the UV Germicidal Lamp 60 the UV radiation emitted kills the viruses and bacteria on the surface of each handrail 401.

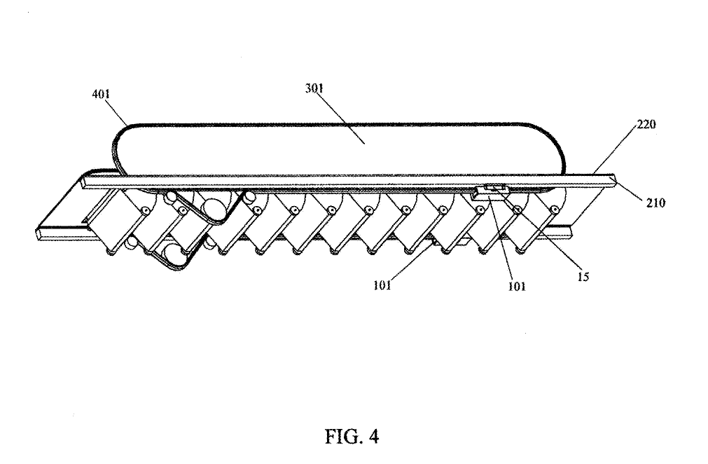

FIG. 4 shows the UV Germicidal Lamp Housing 101 mounted to the moving walkway substructure 105 for each handrail 401 on each side of the moving walkway 301 with a mounting bracket 15. The unit is positioned beneath the fascia panel 220 in the service area of the moving walkway 301. Every time the handrails 401 passes through the UV Germicidal Lamp Housing 101 and is exposed to the UV Germicidal Lamp 60 the UV radiation emitted kills the viruses and bacteria on the surface of each handrail 401.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A system for sterilization of handrails comprising:
providing an UV light sterilization unit;
identifying a placement position for said unit, said position being proximately located to a moving escalator or walkway handrail;
placing said unit in said placement position;
activating said UV light unit, wherein said activation sterilizes said handrail continually while said handrail is in motion.

2. The system of claim 1 wherein said unit provides UV light at a wavelength from 240-280 nm.

3. The system of claim 1 wherein said unit provides UV light at a wavelength of 254 nm.

4. The system of claim 1 wherein said unit provides continuous UV light.

5. The system of claim 1 wherein said unit provides UV light in regular pulses.

6. The system of claim 1 wherein said unit provides a degree of microbial inactivation by light intensity and exposure time at 2,000 to 8,000 μW·s/cm².

7. The system of claim 6 wherein said intensity and exposure time is correlated such that said unit provides said intensity and exposure time relative to said moving handrail.

8. The system of claim 1 wherein said placement provides an unobstructed light pathway to said handrail.

9. The system of claim 1 wherein said placement is in a position where it is not accessible to users of said handrail.

10. The system of claim 1 wherein said unit further includes detection means to measure the level of microorganisms on said handrail.

11. The system of claim 1 wherein said unit further includes detection means to measure the level of microorganisms on said handrail and automatic adjustments of UV light duration and intensity based on said measurements.

12. The system of claim 1 wherein said unit further comprises means to measure the speed of said handrail.

13. The system of claim 1 wherein said unit further comprises means to measure the speed of said handrail and adjusts light and intensity based on said speed.
